(19) United States
(12) Patent Application Publication
(10) Pub. No.: US 2024/0316553 A1
(43) Pub. Date: Sep. 26, 2024

(54) DETECTION SYSTEM

(71) Applicants: Beijing BOE Sensor Technology Co., Ltd., Beijing (CN); BOE Technology Group Co., Ltd., Beijing (CN)

(72) Inventors: Le GU, Beijing (CN); Wenliang YAO, Beijing (CN); Yingying ZHAO, Beijing (CN); Bolin FAN, Beijing (CN); Qiuxu WEI, Beijing (CN); Yongjia GAO, Beijing (CN); Li YANG, Beijing (CN)

(21) Appl. No.: 18/578,682

(22) PCT Filed: Sep. 29, 2021
(86) PCT No.: PCT/CN2021/121892

§ 371 Date: Jan. 11, 2024

(51) Int. Cl. B01L 3/00

(52) U.S. Cl. CPC B01L 3/502761; B01L 2200/0668; B01L 2400/0415

(57) ABSTRACT

A detection system applied to detection of microfluidic chips, includes: a detection chip including a base substrate, an electrode layer and a microfluidic channel layer for accommodating a sample solution having magnetic beads, the base substrate is provided with a bearing surface, the electrode layer is on the bearing surface, the microfluidic channel layer is on the side of the electrode layer away from the base substrate, the electrode layer includes electrodes including at least one strong magnetic electrode and driving electrodes; a magnetic field device being on the side of the base substrate away from the electrode layer, and having a strong magnetic region corresponding one to one to the strong magnetic electrode; a driving mechanism being connected to the magnetic field device, and driving the magnetic field device to approach or move away from the detection chip in a direction that is perpendicular to the bearing surface.

### CROSS REFERENCE TO RELATED APPLICATION

The present disclosure is a National Stage of International Application No. PCT/CN2021/121892, filed on Sep. 29, 2021. The entire contents of which are incorporated herein by reference.

### TECHNICAL FIELD

The disclosure relates to the technical field of biomedical technology, particularly to a detection system applied to a microfluidic chip.

### BACKGROUND

Research on microfluidic chips began in the early 1990s, presenting a potential technology for lab-on-a-chip. Microfluidic chips integrate the sample solution preparation, reaction, separation, and detection processes of biological, chemical, and medical analyses into a single chip at the micrometer scale. The microchannels form a network, allowing controlled fluid flow throughout the system, replacing various functions of conventional biological or chemical laboratories and automating the entire analysis process.

Digital microfluidics (DMF) is a powerful technology used for the precise manipulation of microscale droplets. Based on the dielectric wetting principle, DMF enables the electrical control of individual discrete liquid droplets. In biological analyses based on digital microfluidics, such as library preparation and gene sequencing, precise manipulation of various particles in droplets, including purification, separation, size selection, and enrichment, is often required.

### SUMMARY

The disclosure provides the following technical solution.

A detection system for microfluidic chip detection, includes a detection chip. The detection chip includes a base substrate, an electrode layer, and a microfluidic channel layer for accommodating a sample solution with magnetic beads; the base substrate has a bearing surface, and the electrode layer is formed on the bearing surface; the microfluidic channel layer is disposed on a side of the electrode layer away from the base substrate; the electrode layer includes a plurality of electrodes, and the plurality of electrodes comprises at least one strong magnetic electrode and multiple driving electrodes. The detection system further includes a magnetic field device. The magnetic field device is disposed on a side of the base substrate away from the electrode layer and has a strong magnetic zone corresponding to the strong magnetic electrode. The detection system further includes a drive mechanism. The drive mechanism is connected with the magnetic field device and drives the magnetic field device to move towards or away from the bearing surface of the base substrate. In response to the magnetic field device being in a working position close to the detection chip, for each corresponding pair of strong magnetic zone and strong magnetic electrode, the strong magnetic zone is configured for causing magnetic beads in the sample solution on a side of the strong magnetic electrode away from the base substrate to gather; in response to the magnetic field device being in a working position away from the detection chip, causing the magnetic beads in the sample solution on a side of the strong magnetic electrode away from the base substrate to disperse.

Optionally, the magnetic field device includes a fixing body and a plurality of permanent magnets. The fixing body has a side-open mounting groove, including a bottom wall, a first side wall, a second side wall, a third side wall, and a fourth side wall. The first side wall and the second side wall are opposite, and the first side wall is located at a side of the second side wall facing the base substrate. The third side wall and the fourth side wall are opposite and arranged along the first direction, where the first direction is parallel to the bearing surface of the base substrate. The first side wall has a first opening corresponding to the strong magnetic electrode in a one-to-one manner, allowing a magnetic field to pass through to form the strong magnetic zone. The plurality of permanent magnets are installed in the mounting groove, and arranged along the first direction.

Optionally, the detection system further includes a pressing component. At least one of the third side wall and the fourth side wall has a second opening that passes through its own thickness along the first direction. At least a portion of the pressing component enters into the mounting groove through the second opening, and an entered part of the pressing component abuts against the permanent magnet adjacent to the second opening among the plurality of permanent magnets in the mounting groove, allowing each pair of adjacent permanent magnets to abut against each other.

Optionally, a surface of at least one of the third side wall and the fourth side wall, facing the mounting groove, is provided with an avoidance slot for placing and retrieving the permanent magnets.

Optionally, the second side wall includes a placement slot with an embedded magnet, where the fixing body is magnetically connected to the drive mechanism through the embedded magnet.

Optionally, the second side wall includes multiple placement slots arranged along the first direction on the second side wall.

Optionally, along the first direction, every two adjacent ones among the plurality of permanent magnets have their N pole orientations perpendicular to each other, and the N pole orientations of every two adjacent permanent magnets 23 rotate 90° in a same direction around a rotation axis parallel to the second direction. Here, the second direction is perpendicular to the first direction and parallel to the bearing surface.

Optionally, for each corresponding pair of strong magnetic electrode and the first opening, an orthographic projection of the first opening on the bearing surface is smaller than an orthographic projection of the strong magnetic electrode on the bearing surface, and the orthographic projection of the first opening on the bearing surface is within the orthographic projection of the strong magnetic electrode on the bearing surface.

Optionally, for each corresponding pair of strong magnetic electrode and the first opening, an axis of the first opening is perpendicular to the bearing surface, and the axis of the first opening passes through a center of the strong magnetic electrode.

Optionally, the plurality of electrodes are arranged in an array; and for multiple electrodes along the first direction, each pair of adjacent strong magnetic electrodes has at least one driving electrode.

Optionally, a diameter of the first opening is in a range of 1 mm to 3 mm.

Optionally, the detection system further includes a frame and a pressing structure connected to the frame, where the detection chip is fixed to the frame via the pressing structure.

Optionally, the drive mechanism includes a fixing part, an expansion part, and a support platform. The fixing part is fixed relative to the frame. The expansion part is movably installed on the fixing part along a third direction perpendicular to the bearing surface. The support platform is installed on the expansion part, and the magnetic field device is installed on the support platform.

Optionally, the drive mechanism includes a fixing structure, an expansion component, and a support platform. The fixing structure includes a base and two connection parts, where the base and the two connection parts cooperatively form a U-shaped structure; the two connection parts are fixedly connected to the base and the frame, and the base comprises a through-hole that passes through its thickness along a third direction perpendicular to the bearing surface. The expansion component includes a fixing part and an expansion part, where the fixing part is located at a side of the base away from the support platform and is fixedly connected to the base; the expansion part is movably installed on the fixing part along the third direction, and a free end of the expansion part passes through the through-hole into a space enclosed by the base and the two connection parts. The support platform is located in a space enclosed by the U-shaped structure and is fixedly connected to the free end of the expansion part.

### BRIEF DESCRIPTION OF FIGURES

FIG. 1 is a schematic structural diagram of a detection system provided by embodiments of the disclosure.

FIG. 2 is a sectional view of a detection chip of a detection system provided by embodiments of the disclosure.

FIG. 3 is a magnetic field distribution diagram of a plurality of permanent magnets of a magnetic field device in a detection system provided by embodiments of the disclosure.

FIG. 4 is a stereo view of a magnetic field device in a detection system provided by embodiments of the disclosure.

FIG. 5 is an exploded view of a magnetic field device in a detection system provided by embodiments of the disclosure.

FIG. 6 is a schematic structural diagram of a body of a magnetic field device in a detection system provided by embodiments of the disclosure.

FIG. 7 is a schematic structural diagram of a drive mechanism provided by embodiments of the disclosure.

FIG. 8 is a schematic structural diagram of another drive mechanism provided by embodiments of the disclosure.

FIG. 9A is the first schematic diagram showing a separation process of a sample solution with magnetic beads in a detection system provided by embodiments of the disclosure.

FIG. 9B is the second schematic diagram showing a separation process of a sample solution with magnetic beads in a detection system provided by embodiments of the disclosure.

FIG. 9C is the third schematic diagram showing a separation process of a sample solution with magnetic beads in a detection system provided by embodiments of the disclosure.

FIG. 9D is the fourth schematic diagram showing a separation process of a sample solution with magnetic beads in a detection system provided by embodiments of the disclosure.

FIG. 10A is the first schematic diagram showing an enrichment process of a sample solution with magnetic beads in a detection system provided by embodiments of the disclosure.

FIG. 10B is the second schematic diagram showing an enrichment process of a sample solution with magnetic beads in a detection system provided by embodiments of the disclosure.

FIG. 10C is the third schematic diagram showing an enrichment process of a sample solution with magnetic beads in a detection system provided by embodiments of the disclosure.

FIG. 10D is the fourth schematic diagram showing an enrichment process of a sample solution with magnetic beads in a detection system provided by embodiments of the disclosure

### DETAILED DESCRIPTION

The technical solutions of the embodiments of the disclosure will be clearly and completely described below in conjunction with the accompanying drawings in the embodiments of the disclosure. It is evident that the described embodiment is only a part not all of the embodiments disclosed herein. Based on the embodiments disclosed herein, all other embodiments obtained by those skilled in the art without inventive labor are within the scope of protection of this disclosure.

Digital Microfluidics (DMF) is a powerful technology for the simple and precise manipulation of micro-scale droplets. Digital microfluidics technology is based on the principles of dielectric wetting and allows for the electrical manipulation of individual discrete droplets. By sequentially applying voltage to different electrodes, operations such as movement, separation, and mixing of droplets can be performed on the chip. In contrast to traditional microfluidics technology, the use of electrical driving eliminates the need for external components such as micro-pumps and micro-valves to provide the power for fluid movement. Traditional purification, separation, and enrichment methods for microfluidic chips typically involve handling sample solutions outside the chip using reagents, tubes, centrifuge tubes, etc. Traditional methods consume large amounts of reagents, and the sample solution concentration is often too low for processing and detection on a microfluidic chip without prior purification, separation, and concentration. Performing purification, separation, and concentration of reagents outside the microfluidic chip for each independent sample solution is time-consuming, labor-intensive, and prone to sample solution contamination.

The following is a detailed description of the operation of sample solution B using digital microfluidic technology.

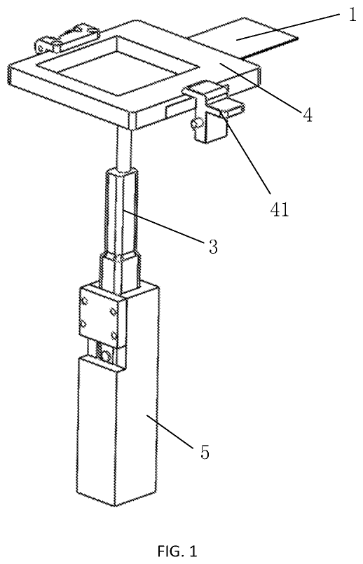

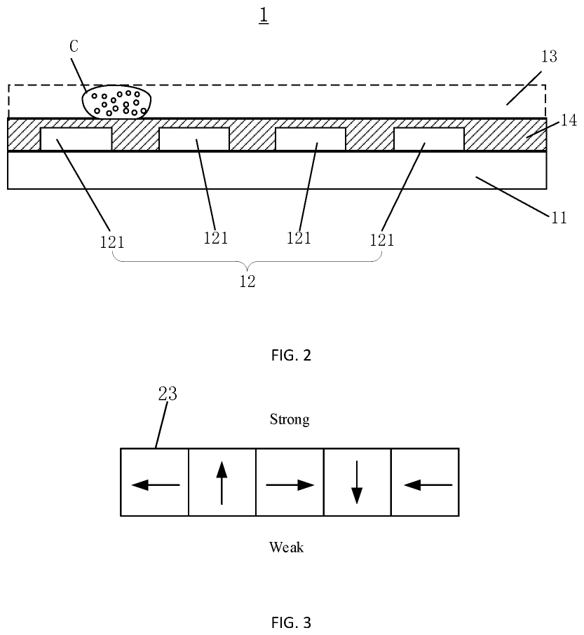

As shown in FIGS. 1 and 2, embodiments of the disclosure provide a detection system for microfluidic chip testing. The detection system includes:
a detection chip 1, including a base substrate 11, an electrode layer 12, a hydrophobic layer 14, and a microfluidic channel layer 13 for accommodating a sample solution B with magnetic beads A; here, the base substrate 11 has a bearing surface, and the electrode layer 12 is formed on the bearing surface of base substrate 11; the microfluidic channel layer 13 is disposed on a side facing away from base substrate 11, of electrode layer 12; the hydrophobic layer 14 is located between the microfluidic channel layer 13 and the electrode layer 12; the electrode layer 12 includes a plurality of electrodes 121, and the plurality of electrodes 121 includes at least one strong magnetic electrode 1211 and multiple driving electrodes 1212; preferably, the plurality of electrodes 121 can be arranged in an array; the electrodes 121 are arranged in the X and Y directions, and the sample solution B with magnetic beads A can move along the X and Y directions through the interaction between electrodes 121;
a magnetic field device 2 on a side away from electrode layer 12, of base substrate 11 and has a strong magnetic zone corresponding to the strong magnetic electrode 1211 in a one-to-one manner; and
a drive mechanism 3 connected with the magnetic field device 2, here the drive mechanism 3 drives the magnetic field device 2 to move towards or away from the bearing surface of base substrate 11 in a direction perpendicular to the base substrate 11;
where in response to the magnetic field device 2 being in the working position close to the detection chip 1, between each corresponding pair of the strong magnetic zone and the strong magnetic electrode 1211, the strong magnetic zone is used to gather the magnetic beads A in the sample solution B on a side of base substrate 11 away from the strong magnetic electrode 1211;
in response to the magnetic field device 2 being in the working position far away from the detection chip 1, the strong magnetic zone releases the magnetic control of the magnetic beads A in the sample solution B on the side of base substrate 11 away from the strong magnetic electrode 1211, causing the magnetic beads A to disperse.

In the use of the detection system described above, firstly, a sample solution B containing a target substance is provided. The sample solution B contains a target substance such as DNA, RNA, etc. The sample solution B is mixed uniformly with magnetic beads A, causing the target substance in the sample solution B to covalently bind to the functional groups on the surface of magnetic beads A, forming a magnetic bead-target substance complex C. The sample solution B mixed with magnetic beads A is injected into the detection chip 1 and fills the microfluidic channel layer 13. When the drive mechanism 3 drives the magnetic field device 2 to move to the position away from the detection chip 1, and controls the driving electrodes 1212 according to the set drive timing, the droplets containing the magnetic bead-target substance complex move in the direction parallel to the base substrate 11, toward the strong magnetic electrode 1211 on the side of the base substrate 11 away from the base substrate 11. Subsequently, the drive mechanism 3 drives the magnetic field device 2 to move to the position close to the detection chip 1, the magnetic beads A in the droplets are fixed on the surface of the strong magnetic electrode 1211 of the detection chip 1 and gather at the strongest magnetic field position, due to the magnetic field of the strong magnetic zone. Then, by controlling the driving electrodes 1212 with the timing of the driving circuit, the sample solution B moves in the direction parallel to the bearing surface of the base substrate 11, allowing the magnetic beads A in the sample solution B that are free from the magnetic field to move to a specific position for fixation, reducing the loss of magnetic beads A in the sample solution B. After fixing the magnetic beads A, continue to control the electrodes 1212 with the timing of the driving circuit, allowing the sample solution B to move in the direction away from the magnetic beads A. The electrode-driven method of manipulating sample solution B can be done with droplet precision, reducing the consumption of the corresponding reagents during the sample solution B and the detection process. After fixing the magnetic beads A, continue to control the electrodes 1212 with the timing of the driving circuit, allowing the sample solution B to move in the direction away from the magnetic beads A. Since the dielectric wetting force in the sample solution B is greater than the resistance of the magnetic beads A fixed on the chip to the droplets, the magnetic bead-target substance complex C is separated from the sample solution B. This achieves the purification, separation, and enrichment of the target sample solution adsorbed on magnetic beads A. This system and method can handle and analyze various sample solutions and find wide applications in the field of biological analysis.

The detection system provided in embodiments of the disclosure uses magnetic beads A and magnetic field device 2 in combination to achieve solid-liquid separation. Specifically, the process involves coating purified sample solution B on the surface of nanoscale biological magnetic beads A. By adsorbing the sample solution B (such as nucleic acids, i.e., DNA or RNA, etc.) onto the surface of magnetic beads A and applying the magnetic field from magnetic field device 2, the nano-magnetic beads A with adsorbed nucleic acids are separated from the liquid, realizing the solid-liquid separation, and realizing the purification, separation, and enrichment of nucleic acids. The described method is simple to operate, provides high extraction purity, is non-toxic, non-polluting, and is suitable for automation and high-throughput operations.

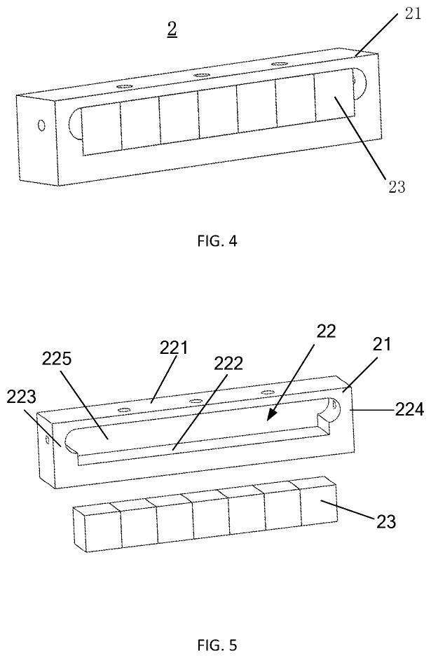

On the basis of the specific embodiments mentioned above, the magnetic field device in the detection system can be configured in various ways, and the specific structure can be set according to practical needs. In one specific embodiment, as shown in FIGS. 4, 5, and 6, the magnetic field device in the detection system includes a fixing body 21 and multiple permanent magnets 23.

The fixing body 21 has an open-sided mounting groove 22, including a bottom wall 225, a first side wall 221, a second side wall 222, a third side wall 223, and a fourth side wall 224. The first side wall 221 and the second side wall 222 are opposite, and the first side wall 221 is located at a side of the second side wall 222 facing the base substrate 11. The third side wall 223 and the fourth side wall 224 are opposite and arranged along a first direction parallel to the bearing surface of base substrate 11. The first side wall 221 has first openings 2211 corresponding to the strong magnetic electrodes 1211 in a one-to-one manner, allowing the magnetic field to pass through to form the strong magnetic zone.

The multiple permanent magnets 23 are installed in the mounting groove 22, and the permanent magnets 23 are arranged along the first direction.

In this magnetic field device 2, each permanent magnet 23 is installed in the mounting groove 22 of the fixing body 21, improving the stability of the relative positions between the permanent magnets 23. Further, strong magnetic zones corresponding to the strong magnetic electrodes 1211 can be formed via the first openings 2211 on the first side wall 221, ensuring good structural stability and stable control of the magnetic field direction in the strong magnetic zones.

To further enhance the stability of the structure of the magnetic field device 2 provided by the technical solution, the permanent magnets 23 can be adhered to each other. The adhered structure of the permanent magnets 23 is then embedded in the mounting groove 22.

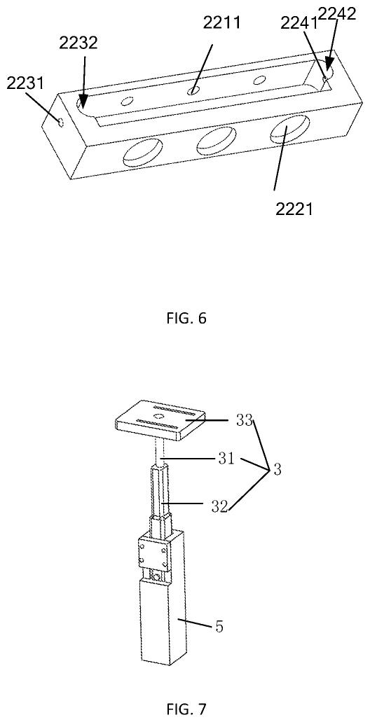

Furthermore, to ensure the stability of the connection between the permanent magnets 23 and the fixing body 21, the magnetic field device 2 includes a pressing component. At least one of the third side wall 223 and the fourth side wall 224 has at least one second opening that traverses its own thickness along the first direction. For example, as shown in FIG. 6, the third side wall 223 has a second opening 2231, and the fourth side wall has a second opening 2241. Taking the second opening 2231 in the third side wall 223 as an example, a portion of the pressing component enters into the mounting groove 22 through the second opening 2231 and an entered part of the pressing component abuts against the permanent magnet 23 adjacent to the second opening among the plurality of permanent magnets in the mounting groove 22, allowing each pair of adjacent permanent magnets 23 to abut against each other.

Of course, to facilitate the assembly between the permanent magnets 23 and the fixing body 21, and to ensure a more secure fixation of the permanent magnets 23, in another embodiment, a pressing component is not included. Only the third side wall 223 has a second opening 2231, and/or the fourth side wall 224 has a second opening 2241. When installing the permanent magnets 23, a push rod can be used to push the permanent magnets 23 in the mounting groove 22 tight along the first direction through the second opening 2231 and/or the second opening 2241.

To facilitate the assembly or disassembly of the permanent magnets 23 in the mounting groove 22, in some embodiments, as shown in FIG. 6, a surface of at least one of the third side wall 223 and the fourth side wall 224 has an avoidance slot on a surface facing the mounting groove 22. As shown in FIG. 6, the third side wall 223 has an avoidance slot 2232, and the fourth side wall 224 has an avoidance slot 2242. The avoidance slot facilitates the insertion of magnetic fixtures or hands into the mounting groove 22 to assemble or disassemble the permanent magnets 23.

On the basis of the various embodiments mentioned above, to facilitate the assembly or disassembly between the magnetic field device 2 and the drive mechanism 3, in some embodiments, as shown in FIG. 6, the second side wall 222 of the mounting groove 22 has placement slots 2221. The placement slots 2221 contain magnets (not shown in the figure), and the fixing body 21 is magnetically connected to the drive mechanism 3 based on the magnetic force of the magnets. Further, based on the magnetic connection between the fixing body 21 and the drive mechanism 3, the specific position of the magnetic field device 2 can be changed flexibly, thus enabling a wider application.

Moreover, for increased stability in the magnetic connection between the fixing body 21 and the drive mechanism 3, multiple placement slots 2221 along the first direction are arranged.

As the embodiments of the disclosure provides a detection chip 1 as a digital microfluidic chip, which is relatively small in size, using multiple independent permanent magnets 23 in combination may result in cross-interference of magnetic fields when handling multiple sample solutions B. In the embodiments, radial and parallel arrangements of multiple permanent magnets 23 are combined. Specifically, as shown in FIGS. 3, 4, and 5, several permanent magnets 23 are installed in the mounting groove 22 along the length direction of the mounting groove 22. Preferably, as shown in FIG. 3, along the first direction, every two adjacent ones among the several permanent magnets 23 have their N pole orientations perpendicular to each other, and the N pole orientations of every two adjacent permanent magnets 23 rotate 90° in the same direction around a rotation axis parallel to the second direction. The second direction is perpendicular to the first direction and parallel to the bearing surface of the base substrate 11. When arranged in this way, the magnetic lines of the permanent magnets 23 converge at a side facing the electrodes 121, resulting in significantly enhanced magnetic force at this side and weakened magnetic force on the other side, thereby obtaining a strong one-sided magnetic field.

Further, for each pair of corresponding strong magnetic electrode 1211 and the first opening 2211, an orthographic projection of the first opening 2211 on the bearing surface of the base substrate 11 is smaller than an orthographic projection of the strong magnetic electrode 1211 on the bearing surface, and the orthographic projection of the first opening 2211 on the bearing surface is located within the orthographic projection of the strong magnetic electrode 1211. In this case, the setting of the first opening 2211 is conducive to further focusing the magnetic field in the strong magnetic zone corresponding to the strong magnetic electrode 1211 and prevents mutual interference of magnetic fields in the strong magnetic zones corresponding to different strong magnetic electrodes 1211.

Specifically, the diameter of the first opening 2211 is in the range of 1 mm-3 mm.

Preferably, to ensure that the magnetic field device 2 can better gather the magnetic beads A, between each pair of corresponding strong magnetic electrode 1211 and the first opening 2211, an axis of the first opening 2211 is perpendicular to the bearing surface, and the axis of the first opening 2211 passes through a center of the strong magnetic electrode 1211.

Referring to FIG. 1 again, the detection system provided in embodiments of the disclosure further includes a frame 5 and a pressing structure 4 connected with the frame 5, where the pressing structure 4 fixes the detection chip 1 to the frame 5. When the detection chip 1 is to be fixed or removed, only the pressing structure 4 needs to be opened, the detection chip 1 is inserted into the pressing structure 4. In order to ensure the stability of the detection chip 1 in the pressing structure 4, the pressing structure 4 is fixed to the detection platform on the frame 5 by a fixing piece 41.

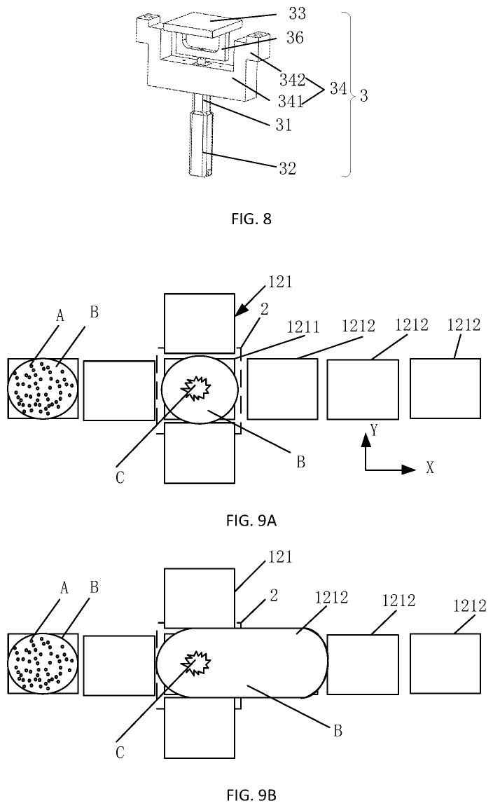

As shown in FIGS. 7 and 8, regarding the drive mechanism 3, there are multiple options, including the following.

Option 1: as shown in FIG. 7, the drive mechanism 3 includes a fixing part 32, an expansion part 31, and a support platform 33.

The fixing part 32 is fixed relative to the frame 5.

The expansion part 31 is movably installed along a third direction on the fixing part 32, where the third direction is perpendicular to the bearing surface.

The support platform 33 is installed on the expansion part 31, and the magnetic field device (not shown in the figure) is installed on the support platform 33. The magnets placed in the placement slots 2221 on the second side wall 222 of the magnetic field device 2 are magnetically connected to the support platform 33. When it is necessary to gather magnetic beads A in the detection chip 1, the movement of the expansion part 31 drives the magnetic field device 2 closer to the detection chip 1, thereby aggregating the magnetic beads A on the detection chip 1. When it is necessary to disperse the magnetic beads A, the movement of the expansion part 31 drives the magnetic field device 2 away from the detection chip 1. The specific expansion part and fixing part can be an electric push rod or a cylinder, thus achieving the effect of expansion and contraction.

Option 2: the drive mechanism 3 includes a fixing structure 34, an expansion component, and a support platform 33.

The fixing structure 34 includes a base 341 and two connection parts 342. The base 341, in cooperation with the two connection parts 342, forms a U-shaped structure. The two connection parts 342 are fixedly connected to the base 341 and the frame (here the frame refers to the operating platform of the detection system, which is not shown in the figure), and the base 341 has a through-hole running through its thickness along a third direction perpendicular to the bearing surface.

The expansion component includes a fixing part 32 and an expansion part 31. The fixing part 32 is located at a side of the base 341 away from the support platform (here, the support platform refers to the operating platform of the detection system, not shown in the figure), and is fixedly connected to the base 341. The expansion part 31 can be movably installed along the third direction on the fixing part 32, and the free end of the expansion part 31 passes through the through-hole into the space enclosed by the base 32 and the two connection parts 342.

The support platform 33 is located in the space enclosed by the U-shaped structure and is fixedly connected to the free end of the expansion part 31. To ensure the stability of the support platform 33, a U-shaped connector 36 is set between the free end of the expansion part 31 and the support platform, and the two ends of the U-shaped connector 36 are connected to the support platform. Of course, the specific U-shaped connector can have other structures, and there are no specific restrictions here.

The usage process of the detection system provided in embodiments of the disclosure is described in detail below.

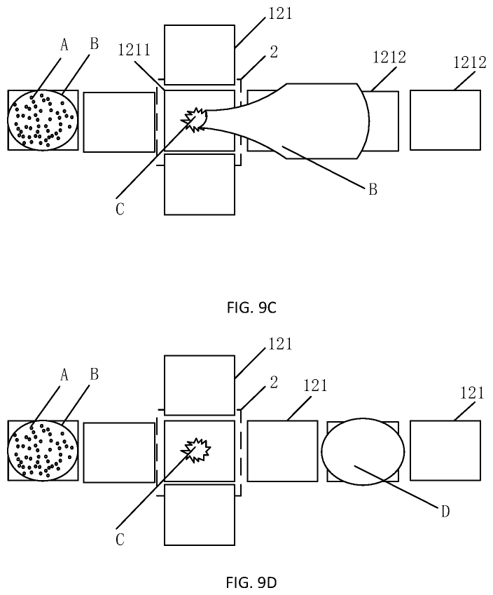

As shown in FIGS. 9A-9D, first, the sample solution B and magnetic beads A are generated from their respective storage tanks, fused and uniformly mixed, and incubated. This allows the target substance in the sample solution B to bind to the magnetic beads A, forming magnetic bead-target substance complexes C. The sample solution B mixed with magnetic beads A is injected into the detection chip 1 and fills the microfluidic channel layer 13. When the drive mechanism 3 drives the magnetic field device 2 away from the detection chip 1, the liquid droplets containing the magnetic bead-target substance complexes C, under the control of the drive electrode 1212's driving timing, allow the sample solution B with magnetic beads A to move to a specific position along the X and Y directions. This specific position is the strong magnetic position of the magnetic field device 2, that is, the position of the strong magnetic electrode 1211. At this time, the drive mechanism 3 drives the magnetic field device 2 closer to the detection chip 1, the magnetic field device 2 rises and attaches to the detection chip 1. The magnetic beads A in the magnetic bead-target substance complexes C are fixed in the strong magnetic zone on the detection chip 1 under the action of the magnetic field. Then, by controlling the timing of the drive circuit to drive the drive electrode 1212, the waste liquid D is moved parallel to the bearing surface of the substrate 11 to separate the liquid droplets from the magnetic bead-target substance complexes C. The liquid droplets of waste liquid D move to the waste liquid tank. The above process is the separation process of the sample solution B.

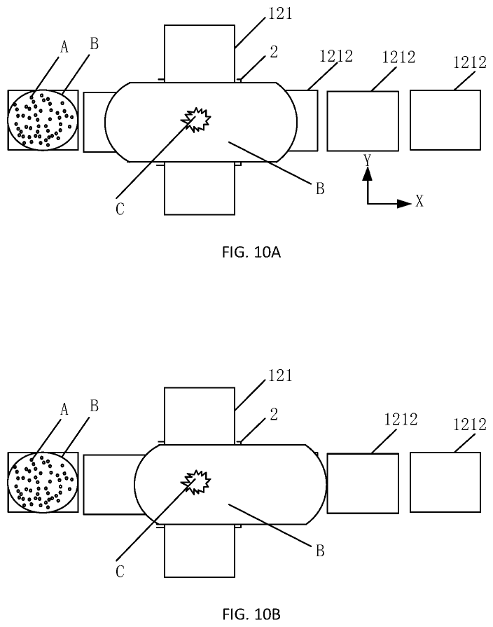

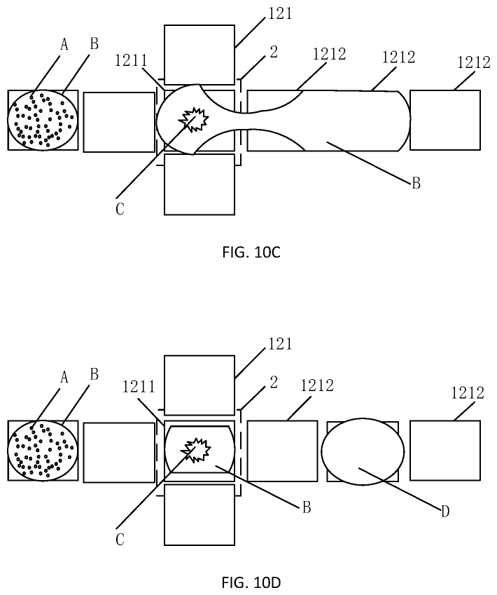

Then, the magnetic bead-target substance complexes C remaining on the electrode 121 are mixed with the washing solution generated in the washing liquid pool. At this point, the drive mechanism 3 controls the magnetic field device 2 to descend. Under the action of the drive electrode 1212, the mixture is oscillated in the X and Y directions, allowing the magnetic bead-target substance complexes C to be suspended. The washing solution is fully mixed with the magnetic bead-target substance complexes C, and unbound target substances and impurities are removed. The drive mechanism 3 controls the magnetic field device 2 to rise, re-fixing the cleaned magnetic bead-target substance complexes C on the chip, and removing the waste liquid D from the mixed solution to the waste liquid tank. This washing step can be repeated 2-3 times. The above process is shown in FIGS. 10A-10D.

Finally, the magnetic bead-target substance complexes C fixed on the chip continue to mix with the elution solution generated in the elution liquid pool. At this point, the magnetic field device 2 descends, and the mixture is oscillated in the X and Y directions to suspend the magnetic beads A in the magnetic bead-target substance complexes. The elution solution is fully mixed with the magnetic bead-target substance complexes C, causing the target substance in the magnetic bead-target substance complexes C to separate from the magnetic bead A and disperse into the elution solution. At this time, the magnetic field device 2 rises, fixing the magnetic beads A on the chip, moving the elution solution for solid-liquid separation, and collecting the elution solution at the sample outlet. The elution solution is the purified sample solution B obtained through the above series of processes, completing the purification, separation, and enrichment of the sample solution B.

Apparently, those skilled in the art can make various modifications and variations to the embodiments of the disclosure without departing from the spirit and scope of the embodiments of the disclosure. In this way, if these modifications and variations of the embodiments of the disclosure fall within the scope of the claims of the disclosure and their equivalent technologies, the disclosure is also intended to include these modifications and variations.

1. A detection system applied to microfluidic chip detection, comprising:
a detection chip, comprising a base substrate, an electrode layer, and a microfluidic channel layer for accommodating a sample solution with magnetic beads;
wherein
the base substrate comprises a bearing surface, and the electrode layer is formed on the bearing surface;
the microfluidic channel layer is disposed on a side away from the base substrate, of the electrode layer;
the electrode layer comprises a plurality of electrodes, and the plurality of electrodes comprises at least one strong magnetic electrode and multiple driving electrodes;
a magnetic field device disposed on a side away from the electrode layer of the base substrate and comprising a strong magnetic zone corresponding to the strong magnetic electrode in a one-to-one manner;
a drive mechanism connected with the magnetic field device, and driving the magnetic field device to move towards or away from the detection chip in a direction vertical to the bearing surface;
wherein in response to the magnetic field device being in a working position close to the detection chip, for each corresponding pair of strong magnetic zone and strong magnetic electrode, the strong magnetic zone is configured for causing magnetic beads in the sample solution on a side away from the base substrate, of the strong magnetic electrode to gather;
in response to the magnetic field device being in a working position away from the detection chip, causing the magnetic beads in the sample solution on a side away from the base substrate, of the strong magnetic electrode to disperse.

2. The detection system according to claim 1, wherein the magnetic field device comprises a fixing body and a plurality of permanent magnets;
wherein
the fixing body comprises a side-open mounting groove comprising a bottom wall, a first side wall, a second side wall, a third side wall, and a fourth side wall; wherein
the first side wall and the second side wall are opposite, and the first side wall is located at a side facing the base substrate, of the second side wall;
the third side wall and the fourth side wall are opposite and arranged along a first direction, wherein the first direction is parallel to the bearing surface of the base substrate; and
the first side wall comprises a first opening corresponding to the strong magnetic electrode in a one-to-one manner, allowing a magnetic field to pass through to form the strong magnetic zone;
the plurality of permanent magnets are installed in the mounting groove, and arranged along the first direction.

3. The detection system according to claim 2, further comprising a pressing component;
wherein at least one of the third side wall and the fourth side wall comprises a second opening that passes through its own thickness along the first direction;
at least a portion of the pressing component enters into the mounting groove through the second opening, and an entered part of the pressing component abuts against the permanent magnet adjacent to the second opening among the plurality of permanent magnets in the mounting groove, allowing each pair of adjacent permanent magnets to abut against each other.

4. The detection system according to claim 2, wherein a surface facing the mounting groove, of at least one of the third side wall and the fourth side wall, is provided with an avoidance slot for placing and retrieving the permanent magnets.

5. The detection system according to claim 2, wherein the second side wall comprises a placement slot with an embedded magnet, wherein the fixing body is magnetically connected to the drive mechanism through the embedded magnet.

6. The detection system according to claim 5, wherein the second side wall comprises multiple placement slots arranged along the first direction on the second side wall.

7. The detection system according to claim 2, wherein along the first direction, N pole orientations of every two adjacent ones among the plurality of permanent magnets are perpendicular to each other, and the N pole orientations of every two adjacent permanent magnets rotate 90° in a same direction around a rotation axis parallel to the second direction;
wherein the second direction is perpendicular to the first direction and parallel to the bearing surface.

8. The detection system according to claim 2, wherein for each corresponding pair of strong magnetic electrode and the first opening, an orthographic projection of the first opening on the bearing surface is smaller than an orthographic projection of the strong magnetic electrode on the bearing surface; and
the orthographic projection of the first opening on the bearing surface is within the orthographic projection of the strong magnetic electrode on the bearing surface.

9. The detection system according to claim 8, wherein for each corresponding pair of strong magnetic electrode and the first opening, an axis of the first opening is perpendicular to the bearing surface, and the axis of the first opening passes through a center of the strong magnetic electrode.

10. The detection system according to claim 9, wherein the plurality of electrodes are arranged in an array; and
for multiple electrodes along the first direction, at least one driving electrode is disposed between each pair of adjacent strong magnetic electrodes.

11. The detection system according to claim 9, wherein a diameter of the first opening is in a range of 1 mm to 3 mm.

12. The detection system according to claim 1, further comprising a frame and a pressing structure connected to the frame, wherein the detection chip is fixed to the frame via the pressing structure.

13. The detection system according to claim 12, wherein the drive mechanism comprises a fixing part, an expansion part, and a support platform;
wherein
the fixing part is fixed relative to the frame;
the expansion part is movably installed on the fixing part along a third direction perpendicular to the bearing surface;
the support platform is installed on the expansion part, and the magnetic field device is installed on the support platform.

14. The detection system according to claim 12, wherein the drive mechanism comprises a fixing structure, an expansion component, and a support platform;
wherein
the fixing structure comprises a base and two connection parts, wherein the base and the two connection parts cooperatively form a U-shaped structure; the two connection parts are fixedly connected to the base and the frame, and the base comprises a through-hole that passes through the base's thickness along a third direction perpendicular to the bearing surface;
the expansion component comprises a fixing part and an expansion part, wherein the fixing part is located at a side away from the support platform, of the base and is fixedly connected to the base; the expansion part is movably installed on the fixing part along the third direction, and a free end of the expansion part passes through the through-hole into a space enclosed by the base and the two connection parts;
the support platform is located in a space enclosed by the U-shaped structure and is fixedly connected to the free end of the expansion part.
